(19) United States
(12) Patent Application Publication
(10) Pub. No.: US 2025/0107934 A1
(43) Pub. Date: Apr. 3, 2025

(54) WOUND DRESSING WITH ARC-SHAPED INCISIONS

(71) Applicant: Lohmann & Rauscher GmbH & Co. KG, Neuwied (DE)

(72) Inventor: Daniel Schulze, Siegburg (DE)

(21) Appl. No.: 18/729,814

(22) PCT Filed: Jan. 24, 2023
(86) PCT No.: PCT/EP2023/051666

§ 371 Date: Jul. 17, 2024

(30) Foreign Application Priority Data
Jan. 24, 2022 (DE) 202022100372.6

(51) Int. Cl. A61F 13/0206

(52) U.S. Cl. CPC A61F 13/0209

(57) ABSTRACT

In a wound dressing with a nonwoven comprising fibers, wherein a majority of the fibers are aligned in a machine direction of the nonwoven, wherein the nonwoven has at least one incision, an improvement is presented in which the incision is an arc-shaped incision with a radius of curvature in a range of 0.5 mm to 20 mm, and a tangent applied to the arc-shaped incision has an angle of 30° to 150°, in some embodiments 45° to 135°, and in some embodiments 60° to 120° to the machine direction of the nonwoven.

### CROSS REFERENCE TO RELATED APPLICATION

The present application is a 35 U.S.C. § 371 national phase entry application of, and claims priority to, International Patent Application No. PCT/EP2023/051666, filed Jan. 24, 2023, which claims priority to German Patent Application No. DE 202022100372.6, filed Jan. 24, 2022, the disclosures of which are hereby incorporated by reference in their entirety for all purposes.

### TECHNICAL FIELD

Embodiments relate to a wound dressing with arc-shaped incisions, a wound bandage, a wound care kit and uses of the wound dressing, the wound bandage and the wound care kit.

### BACKGROUND

Modern wound dressings are usually produced from a multiplicity of materials in order to form a multilayer wound dressing which enables improved exudate management. These materials include films, adhesives, foams and fibrous nonwoven. The adaptability of a wound dressing to the wound body site is generally determined by the mechanical properties of its constituents. Some of the known wound dressings contain a nonwoven fabric layer in order to improve the absorption properties of the wound dressing and retention of exudate. However, a disadvantage of nonwoven fabrics is that they have limited extensibility.

Reduced extensibility of wound dressings can cause problems during use, in particular when the wound dressings are applied at sites where high shear stresses act on the wound dressings, for example by bending a knee or an elbow. Limited extensibility of wound dressings can lead, for example, to the wound dressings having poor wearing comfort, the wound dressings partially detaching from the wound, the functionality of the wound dressings being reduced by a lack of wound coverage, and/or even additional lesions of the tissue, such as blisters or skin cracks, being produced by mechanical stressing of the tissue.

A wound dressing according to EP 3 085 344 B1 comprises a nonwoven fabric layer which has discrete groups of rectilinear incisions, wherein each group of incisions comprises at least a first and a second incision which extend from a common starting point to end points spaced apart from one another. According to EP 3 085 344 B1, a wound dressing becomes more flexible due to the presence of discrete groups of incisions, so that the wound dressing can better adapt to the body of the user and the wound dressing is under lower mechanical stress. According to EP 3 085 344 B1, the incisions of a group of incisions are arranged, for example, in a V shape or in the form of a star. However, at the common starting points of a first and second incision of a group of rectilinear incisions, detachment of fibers and parts of the nonwoven fabric layer from the nonwoven occurs, in particular when the first and second incision are not arranged collinearly. Detachment of fibers and parts of the nonwoven fabric layer, in particular at the common starting point of the first and second incision, can result in a limitation of the functionality of the wound dressing. When the known wound dressing is used, the wound healing is therefore still impaired in many cases. Furthermore, it has been found that the fibers and parts of the nonwoven fabric layer detached from the nonwoven fabric can penetrate into the wound. Corresponding nonwoven fabric residues in the wound can disrupt the wound healing. The wound healing process is therefore impaired in many cases despite stress relief of the wound dressing.

A wound care article according to DE 10 2011 002 268 A1 comprises a nonwoven fabric or airlaid material which has a pattern of incisions and/or punchings in order to increase the entry of liquid into the wound care article. According to DE 10 2011 002 268 A1, the entry of liquid into the wound care article is increased by corresponding incisions and/or punchings. However, it has been found that the increased entry of liquid and the corresponding incisions or punchings can lead to the wound care article detaching from the wound and the wearing duration of the wound care article being shortened. Furthermore, it has been found that when the wound care article is used at body sites where high shear stress acts on the wound care article, for example by bending a knee or an elbow, the functionality of the wound care article can be limited by partial detachment from the wound. Furthermore, it has been observed in many cases that corresponding incisions and/or punchings which remove material from the nonwoven fabric result in disintegration of the wound care article, as a result of which the absorptivity and functionality of the wound care article are reduced. Despite increased entry of liquid into the absorptive material of the wound care article, the wound healing process can therefore be impaired and the wearing comfort for the user can be reduced.

### SUMMARY

In view of the above-described problems, embodiments may provide a wound dressing which enables improved wound healing.

In embodiments may achieve the above object through use of one or more nonwoven fabrics (also referred to as “nonwoven”) of a wound dressing that have at least one arc-shaped incision with a radius of curvature in a range of 0.5 millimeters (mm) to 20 mm, and a tangent applied to the arc-shaped incision has an angle of 30 degrees (°) to 150°, and in some embodiments 45° to 135°, and in some embodiments 60° to 120° to the machine direction of the nonwoven. The planar extent of the wound dressing comprises the machine direction and a direction orthogonal thereto, wherein the fibers of the nonwoven have a preferred direction along the machine direction, i.e. a majority of the fibers of the nonwoven are aligned in the machine direction of the nonwoven.

Generally, wound healing with the legacy wound dressings maybe inadequate despite stress relief by incisions or punchings. It has been found that arc-shaped incisions with a radius of curvature in a range of 0.5 mm to 20 mm, and a tangent applied to the arc-shaped incision, which has an angle of 30° to 150°, 45° to 135°, or 60° to 120° to the machine direction of the nonwoven, achieve better wound healing results. Surprisingly, it has been found that particularly many fibers can be cut in the machine direction of a nonwoven by means of arc-shaped incisions, as a result of which excellent stress relief and extensibility of the nonwoven can be brought about. Furthermore, the absorptivity of the wound dressing by means of arc-shaped incisions is maintained and nonwoven fabric residues in the wound are avoided. The wound healing process is thus optimally promoted with a wound dressing according to embodiments. In the context of embodiments herein, incisions are incisions into a material, for example into the nonwoven, without material being removed, because the incisions, in contrast to punchings, do not completely run around a region of the nonwoven. Compared with punchings in which material is removed, incisions have the advantage that disintegration of the material, for example of the nonwoven, does not occur. Punchings in which nonwoven fabric material is removed, such as, for example, circular punchings, have the disadvantage that disintegration of the nonwoven and thus a reduced absorptivity of corresponding wound dressings can occur. It has been found that incisions in which substantially no material is removed from the nonwoven are particularly expedient for wound care, since the nonwoven remains intact and good absorptivity is thus ensured. In some embodiments, a maximum distance between the opposite sides of the arc-shaped incision running along the longest extent of the arc-shaped incision, or the width of the incision, is ≤1 mm. This maximum distance relates in particular to a state in which the wound dressing is not applied to the wound.

In embodiments, for improving the extensibility of the nonwoven in the machine direction it is foreseen that the at least one arc-shaped incision of the nonwoven, for example each arc-shaped incision of a multiplicity of arc-shaped incisions of the nonwoven, has a radius of curvature in a range of 1 mm to 17 mm, or in some embodiments 2 mm to 15 mm, or in some embodiments 3 mm to 10 mm. A greater curvature of the arc-shaped incisions has the advantage that the arc-shaped incisions can be arranged on the wound dressing in such a way that adjacent arc-shaped incisions have as small a distance from one another as possible. An arrangement of adjacent arc-shaped incisions with a small distance makes it possible for many of the fibers running in the machine direction to be severed by the adjacent arc-shaped incisions, as a result of which increased flexibility in the machine direction is ensured. If the nonwoven comprises more than one arc-shaped incision, the arc-shaped incisions of the nonwoven are spaced apart from one another.

In one embodiment, the wound dressing is characterized in that the at least one arc-shaped incision of the nonwoven has a length in a range of 1 mm to 15 mm, or more particularly 2 mm to 10 mm. In some embodiments, all arc-shaped incisions of the nonwoven have a length in a range of 1 mm to 15 mm, or more particularly 2 mm to 10 mm. Such a length of the at least one arc-shaped incision has the advantage that, as a result of the severing of the fibers along the length of the arc-shaped incision, the extensibility of the wound dressing in the machine direction of the nonwoven is increased and the wound dressing nevertheless has a high structural integrity. In some embodiments, the nonwoven has a multiplicity of arc-shaped incisions with a length in a range of 1 mm to 15 mm, or more particularly 2 mm to 10 mm.

With regard to the structural integrity of the nonwoven and the avoidance of discontinuities, it has proven expedient for a length between a first end point of the arc-shaped incision and a second end point of the arc-shaped incision to be embodied in a range of 0.5 mm to 12 mm, or more particularly 1 mm to 9 mm. It has been found that, as a result of the avoidance of discontinuities, detachment of fibers or parts of the nonwoven fabric layer which impairs the wound healing can be avoided.

A straight line running through the first end point of the arc-shaped incision and the second end point of the arc-shaped incision can have an angle of 30° to 150°, or in some embodiments 45° to 135°, or in particular embodiments 60° to 120° to the machine direction of the nonwoven. This has the advantage that the arc-shaped incision severs a multiplicity of the fibers of the nonwoven orthogonally to the fiber orientation, as a result of which the extensibility of the nonwoven in the machine direction is improved.

In terms of production technology and in the sense of improved extensibility of the nonwoven, it has proven to be favorable if the arc-shaped incision of the nonwoven is mirror-symmetrical with respect to a plane running perpendicular to a planar extension of the wound dressing and/or doubly rotationally symmetrical with respect to an axis running perpendicular to the planar extension of the wound dressing. In some embodiments, the nonwoven has a multiplicity of such mirror-symmetrical and/or doubly rotationally symmetrical arc-shaped incisions. Mirror-symmetrical and/or doubly rotationally symmetrical arc-shaped incisions can be present, for example, in the form of a circle segment arc, S-shaped or wave-shaped. If the arc-shaped incision is embodied doubly rotationally symmetrically, the arc-shaped incision is imaged on itself by a rotation through 180°.

As already addressed above, the arc-shaped incision can be embodied as a circle segment arc-shaped incision, S-shaped incision or wave-shaped incision. In this case, it has proven to be expedient with regard to the production of the wound dressing if all arc-shaped incisions of a multiplicity of arc-shaped incisions have an identical shape. The arc-shaped incisions of the multiplicity of arc-shaped incisions can have a different spatial orientation along the planar extent of the nonwoven, in particular along the machine direction and/or the direction orthogonal to the machine direction, and can be converted into one another by symmetry operations, for example mirroring, parallel displacement and/or rotation. For example, the individual arc-shaped incisions of a multiplicity of arc-shaped incisions can have the same shape, but can be present mirrored, displaced parallel and/or rotated with respect to one another.

In one embodiment, the nonwoven has a multiplicity of arc-shaped incisions which are spaced apart from one another. As a result of the presence of a multiplicity of arc-shaped incisions in the nonwoven, many fibers are severed in the machine direction and the extensibility of the nonwoven in the machine direction is increased, so that the wound dressing can better adapt to the body of the user. Surprisingly, it has been found that the functionality of a wound dressing according to embodiments with a multiplicity of arc-shaped incisions is ensured even when used at body sites where high shear stress acts on the wound dressing, since the arc-shaped incisions enable optimal adaptation to corresponding body sites. Advantageously, the arc-shaped incisions of the wound dressing may increase the wearing comfort for the user by reducing the mechanical stressing of the wound while simultaneously avoiding nonwoven fabric residues in the wound.

In the sense of particularly advantageous extensibility of the nonwoven in the machine direction, it has proven to be favorable if at least two arc-shaped incisions, spaced apart from one another, of the multiplicity of arc-shaped incisions have a spacing in a range of 0.5 mm to 10 mm, or more particularly 1 mm to 5 mm. Such a spacing relates to the minimum distance between two points lying on the respective two arc-shaped incisions spaced apart from one another, i.e. between a first point lying on a first arc-shaped incision and a second point lying on a second incision spaced apart from the first arc-shaped incision. As a result of corresponding spacing of adjacent arc-shaped incisions, a high proportion of the fibers of the nonwoven, in particular a high proportion of the fibers aligned in the machine direction of the nonwoven, is severed, so that the wound dressing has excellent extensibility in the machine direction and discontinuities are avoided. In some embodiments, the multiplicity of arc-shaped incisions of the multiplicity of arc-shaped incisions of the nonwoven, and in some embodiments all arc-shaped incisions of the multiplicity of arc-shaped incisions of the nonwoven, have a spacing in a range of 0.5 mm to 10 mm, or particularly 1 mm to 5 mm, from their respective adjacent arc-shaped incisions.

It has proven to be expedient with regard to the extensibility and the avoidance of discontinuities if at least two arc-shaped incisions, spaced apart from one another, of the multiplicity of arc-shaped incisions have a spacing in a range of 0.5 mm to 10 mm, or more particularly 1 mm to 5 mm, along the machine direction of the nonwoven. Such a spacing between two adjacent arc-shaped incisions along the machine direction of the nonwoven makes particularly advantageous extensibility of the wound dressing possible, since the majority of the fibers, which are arranged in the machine direction of the nonwoven and reduce the extensibility of the nonwoven in the machine direction, are present in severed form. Such a spacing along the machine direction relates to the minimum distance between two points lying on the respective two arc-shaped incisions spaced apart from one another along the machine direction of the nonwoven. In some embodiments, the multiplicity of arc-shaped incisions of the multiplicity of arc-shaped incisions of the nonwoven, particularly in some embodiments all arc-shaped incisions of the multiplicity of arc-shaped incisions of the nonwoven, have a spacing in a range of 0.5 mm to 10 mm, or more particularly 1 mm to 5 mm, from their respective adjacent arc-shaped incisions along the machine direction of the nonwoven. Adjacent arc-shaped incisions are those incisions which are adjacent to one another in the machine direction or transversely to the machine direction, i.e. in the direction orthogonal to the machine direction, of the nonwoven, in particular are directly adjacent to one another without incisions arranged therebetween.

Additionally or alternatively, at least two arc-shaped incisions, spaced apart from one another, of the multiplicity of arc-shaped incisions of the nonwoven can have a spacing in a range of 0.5 mm to 10 mm, or more particularly 1 mm to 5 mm, along a direction orthogonal to the machine direction. This enables particularly good extensibility of the nonwoven along a direction of the planar extent of the wound dressing which runs orthogonally to the machine direction of the nonwoven. Such a spacing along a direction orthogonal to the machine direction relates to the minimum distance between two points lying on the respective two arc-shaped incisions spaced apart from one another along the orthogonal direction, i.e. along the planar extent of the wound dressing in the direction orthogonal to the machine direction of the nonwoven. Preferably, the multiplicity of arc-shaped incisions of the multiplicity of arc-shaped incisions of the nonwoven, and in some embodiments all arc-shaped incisions of the multiplicity of arc-shaped incisions of the nonwoven, have a spacing in a range of 0.5 mm to 10 mm, or more particularly 1 mm to 5 mm, from their respective adjacent arc-shaped incisions along the direction orthogonal to the machine direction.

A wound dressing in some embodiments is particularly extensible when the arc-shaped incisions of the multiplicity of arc-shaped incisions of the nonwoven have a spacing in a range of 0.5 mm to 10 mm, or more particularly 1 mm to 5 mm, from their respective incisions, adjacent in the machine direction, along the machine direction of the nonwoven, and have a spacing in a range of 0.5 mm to 10 mm, or more particularly 1 mm to 5 mm, from their respective incisions, adjacent in the direction orthogonal to the machine direction of the nonwoven, along the orthogonal direction.

In a wound dressing of at least one embodiment, the arc-shaped incisions can be designed such that at least two arc-shaped incisions, spaced apart from one another, of the multiplicity of arc-shaped incisions have an overlap in a length in a range of 0.1 mm to 5 mm, or more particularly 0.2 mm to 2 mm, along the machine direction. Such an overlap of incisions along the machine direction has the consequence that the majority of the fibers aligned in the machine direction, and in some embodiments all of the fibers aligned in the machine direction, are severed at least once, as a result of which the extensibility of the wound dressing is increased.

The arc-shaped incisions of a wound dressing according to embodiments can furthermore be designed such that at least two arc-shaped incisions, spaced apart from one another, of the multiplicity of arc-shaped incisions have an overlap in a length in a range of 0.1 mm to 5 mm, or more particularly 0.2 mm to 2 mm, along the direction orthogonal to the machine direction. Such an overlap of incisions along the direction orthogonal to the machine direction has the consequence that the majority of the fibers aligned in the machine direction, and in some embodiments all of the fibers aligned in the machine direction, are severed at least once in the direction orthogonal to the machine direction, as a result of which the extensibility of the wound dressing is increased. A particularly high extensibility of the wound dressing can be achieved if the arc-shaped incisions are designed such that at least two arc-shaped incisions, spaced apart from one another, have an overlap in the machine direction and at least two arc-shaped incisions, spaced apart from one another, have an overlap in the direction orthogonal to the machine direction. The wound dressing can be designed, for example, such that the wound dressing has a pattern of a multiplicity of incisions, spaced apart from one another, wherein at least two respectively adjacent incisions of the pattern have an overlap along the machine direction and at least two respectively adjacent incisions of the pattern have an overlap along the direction orthogonal to the machine direction. In order to achieve a particularly high extensibility of the wound dressing, such a pattern can be designed, for example, such that a multiplicity of incisions, in some embodiments each incision of a multiplicity of incisions, have an overlap in the machine direction and/or an overlap in the direction orthogonal to the machine direction with at least one adjacent incision.

As already explained above, several arc-shaped incisions of the multiplicity of arc-shaped incisions of the nonwoven can have an identical shape. In some embodiments, at least two arc-shaped incisions, spaced apart from one another, of the multiplicity of arc-shaped incisions, in some embodiments all arc-shaped incisions, spaced apart from one another, of the multiplicity of arc-shaped incisions, have an identical shape, for example a circle segment arc shape, S-shape or wave shape. These can be arranged such that the spaced-apart arc-shaped incisions form a pattern of incisions in the nonwoven. In an embodiment, the wound dressing has a multiplicity of incisions, which have an identical shape, wherein respectively adjacent incisions along the machine direction of the nonwoven have a distance in a range of 0.5 mm to 10 mm, or more particularly 1 mm to 5 mm, along the machine direction, and wherein respectively adjacent incisions along the orthogonal direction have a distance in a range of 0.5 mm to 10 mm, or more particularly 1 mm to 5 mm, along the direction of the planar extent of the wound dressing running orthogonally to the machine direction.

In a wound dressing according to embodiments, the nonwoven can have a peripheral region and a central region, wherein the peripheral region and/or the central region has the multiplicity of arc-shaped incisions. Depending on the application, it has proven to be particularly advantageous if only the peripheral region, only the central region, or both the peripheral region and the central region have arc-shaped incisions. For an application at body sites where high shear stress acts on the wound dressing, for example knees or elbows, it can be particularly favorable for the wearing comfort of the user if the incisions are located both in the peripheral region and in the central region. Thus, the user can bend the knee or the elbow without unpleasant mechanical stressing of the wound or detachment of the wound dressing occurring. In other applications, for example relatively small body sites where different mechanical stresses occur, for example toes, heels and fingers, it can be advantageous if only the peripheral region or only the central region has the arc-shaped incisions, for example in order to compensate for a different mechanical stress at transitions between body sites, such as at a transition between toes and sole of the foot.

In at least one embodiment, the arc-shaped incisions of the multiplicity of arc-shaped incisions are distributed uniformly over the nonwoven. In the case of a nonwoven with uniformly distributed incisions, at least 25%, in some embodiments 50%, and in particular embodiments 75% of the incisions of the multiplicity of arc-shaped incisions are configured in such a way that a distance between respectively adjacent incisions is approximately equal and/or that incisions can be converted into one another by symmetry operations. A symmetry operation can comprise mirroring, parallel displacement and/or rotation. For example, a multiplicity of the incisions of the uniformly distributed incisions have an approximately equal spacing, for example a spacing in a range of 0.5 mm to 10 mm, or more particularly 1 mm to 5 mm, from their respectively adjacent incisions along the machine direction and/or along the direction orthogonal to the machine direction. The nonwoven can be configured in such a way that such a uniform distribution of arc-shaped incisions is present in the peripheral region, in the central region, or over the entire nonwoven both in the peripheral region and in the central region. The arc-shaped incisions can be distributed uniformly over the peripheral region and the central region, for example. A uniform distribution of the arc-shaped incisions enables uniform extensibility of the wound dressing, as a result of which the wound dressing is particularly suitable for body sites where high shear stress acts on the wound dressing. The wound dressing according to embodiments comprises the nonwoven and optionally further layers and/or materials, for example an adhesive film. In one embodiment, the wound dressing consists of the nonwoven comprising fibers.

In at least one embodiment, the nonwoven comprises a superabsorbent polymer. A nonwoven with a superabsorbent polymer has the advantage that the absorption of wound fluid into the wound dressing is increased by the osmotic potential of the superabsorbent polymer. Superabsorbent polymers are plastics which are able to absorb a multiple of their own weight, for example up to 1000 times their own weight, of liquids. Superabsorbent polymers can be, for example, copolymers of acrylic acid and sodium acrylate, wherein the ratio of the two monomers to one another can vary. In addition, a core crosslinker (Core-Cross-Linker, CXL) can be added to the monomer solution, which core crosslinker connects the long-chain polymer molecules formed to one another in places by chemical bridges. As a result of these bridges, the polymer becomes water-insoluble.

In at least one embodiment, the superabsorbent polymer is embodied in particulate and/or fibrous form. A particulate superabsorbent polymer can be present, for example, in the form of a granulate and/or a powder. It has proven to be expedient if the particulate superabsorbent polymer has a particle size in a range of 1 micrometer (μm) to 1000 μm, or in some embodiments 100 μm to 1000 μm. A fibrous superabsorbent polymer can be present, for example, in the form of one or more fibers, a fiber knit, a fiber scrim, a fiber nonwoven and/or a fiber batt. The particulate form, for example granulate or powder form, of a superabsorbent polymer has proven very successful and may be desirable because it can be readily incorporated into a nonwoven. Likewise, the fiber form of a superabsorbent polymer may be desirable since this is a product which is very soft both in the dry state and in the swollen state, which can be modeled and which has low abrasiveness.

Embodiments may further relate to a wound bandage which comprises a wound dressing according to embodiments. A wound dressing according to embodiments is prepared for use in wound care and enables simple use by the user. In some embodiments, a wound dressing according to embodiments is a sterile wound bandage.

In at least one embodiment, the wound bandage comprises a carrier layer, a superabsorbent layer, a layer comprising polyurethane and/or a wound contact layer. The carrier layer promotes the stability of the wound bandage and protects against penetration of substances such as dirt, water and bacteria from the outside into the wound bandage. The carrier layer offers particularly good protection if the carrier layer is embodied to be waterproof. The superabsorbent layer increases the absorption of wound fluid and may be desired in wound dressings which are prepared for moderately to strongly exuding wounds. The superabsorbent layer can be the nonwoven of the wound dressing, which nonwoven comprises a superabsorbent polymer, and/or can be an additional layer of the wound dressing, which additional layer comprises a superabsorbent material, for example a superabsorbent polymer. A wound dressing according to embodiments can have, for example, a superabsorbent layer which is embodied in the form of the nonwoven of the wound dressing with a superabsorbent polymer or in the form of a superabsorbent layer separate from the nonwoven, or the wound dressing can have two superabsorbent layers, wherein a first superabsorbent layer is the nonwoven of the wound dressing with a superabsorbent polymer and a second superabsorbent layer is an additional superabsorbent layer. A superabsorbent layer can comprise, for example, an absorptive material, such as cellulose fibers, in which a superabsorbent polymer, for example a sodium acrylate acrylic acid polymer, is embedded. In order to ensure a high absorptive power of the superabsorbent layer(s), a superabsorbent layer can have superabsorbent polymers with a proportion by weight of ≥15 percent (%) by weight to ≤99% by weight, based on the total weight of the superabsorbent layer. A superabsorbent layer can be arranged in a wound dressing, for example between a carrier layer and the nonwoven. The superabsorbent layer or the superabsorbent layers can comprise a superabsorbent polymer, as a result of which good absorption of exudate is ensured. In terms of production technology, it has proven to be favorable if a superabsorbent polymer of a superabsorbent layer(s) is embodied in particulate and/or fibrous form, since excellent embedding of the superabsorbent material in the wound dressing is thus ensured. A particular advantage of fibrous superabsorbent polymers is that a high structural integrity of the superabsorbent layer(s) is achieved, in which no detachment of superabsorbent polymer from the superabsorbent layer occurs. The layer comprising polyurethane promotes excellent exudate management and offers an optimal wound environment for the wound healing. With regard to good removal of liquid from the wound and reduction of the risk of maceration, it has proven to be particularly favorable if the layer comprising polyurethane comprises polyurethane foam. A wound dressing according to embodiment can be prepared, for example, such that a layer comprising polyurethane is arranged between a wound contact layer and the nonwoven of the wound dressing. The wound contact layer increases the wearing comfort for the user. For example, the wound contact layer minimizes the pain of the patient during the application and reduces the trauma for the wounds and surrounding tissue during the change of wound dressing. It has been found that a wound contact layer comprising silicone is particularly suitable for the tissue during the change of wound dressing with regard to a reduction of the pain of the patient and of the trauma.

Within the scope of embodiments herein, it is also envisaged that the superabsorbent layer, the layer comprising polyurethane and/or the wound contact layer comprise at least one arc-shaped incision, and in some embodiments a multiplicity of arc-shaped incisions. In some embodiments, the at least one arc-shaped incision of the superabsorbent layer, of the layer comprising polyurethane and/or of the wound contact layer, if present, has approximately the same properties as the at least one incision of the nonwoven, for example with regard to radius of curvature, length of the incision, orientation along the machine direction, symmetry, shape, spacing and distribution. It has proven to be expedient if a pattern formed from the arc-shaped incisions in the superabsorbent layer, the layer comprising polyurethane and/or the wound contact layer is identical to the pattern formed from the arc-shaped incisions in the nonwoven. If more than one layer of the superabsorbent layer, the layer comprising polyurethane and the wound contact layer has arc-shaped incisions, these layers may have an identical pattern of incisions. For example, such a pattern of incisions can arise in that several layers of the wound dressing, for example a superabsorbent layer, the nonwoven and a layer comprising polyurethane, are first arranged one on top of the other and subsequently the at least one arc-shaped incision is incised, as a result of which the layers arranged one on top of the other each have the at least one arc-shaped incision. It has been found that it is particularly favorable with regard to the extensibility of the wound dressing if not only the nonwoven has at least one arc-shaped incision, but additionally at least one further layer, for example the superabsorbent layer and/or the layer comprising polyurethane, has at least one arc-shaped incision. According to one embodiment, when the wound contact layer has an extensible material, for example silicone, the wound contact layer is embodied without arc-shaped incisions.

A wound dressing according to embodiments can furthermore have one or more peelable protective films which are peelable from the wound contact layer in order to facilitate the application of the wound dressing. Furthermore, the shape of the wound dressing can be configured in such a way that the shape is adapted to the anatomical requirements of the body site for which the wound dressing is prepared. In this case, a wound dressing can be embodied, for example, in the form of a square, rectangle, circle or oval. For wound care of difficult wound locations, for example of the sacral region, the elbow or the heel, the shape of the wound dressing can be prepared in a targeted manner for the corresponding body site.

Embodiments may also relate to a wound care kit with a wound dressing according to at least one embodiment accommodated sterile in a package. An instruction for using the wound dressing in the treatment of wounds may be provided in and/or on the package. The wound care kit according to embodiments enables simple and safe use of the wound dressing on a wound.

As can be gathered from the above explanation of wound dressings and wound bandages according to embodiments, embodiments may also relate to the use of a wound dressing for producing a wound bandage according to embodiments. For this purpose, the wound dressing can be prepared as a wound bandage, for example by joining the wound dressing to further layers, such as a superabsorbent layer, a layer comprising polyurethane and/or a wound contact layer.

Embodiments may also relate to a wound dressing, a wound bandage, and/or a wound care kit for use in wound care. It has been found that excellent wound care can be ensured if the nonwoven of a wound dressing is embodied with arc-shaped incisions which significantly improve the wearing comfort for the patient.

### BRIEF DESCRIPTION OF THE DRAWINGS

Embodiments are explained below with reference to the drawing, to which explicit reference is made with regard to various details of embodiments and which are not further emphasized in the description. In the drawing:

FIG. 1 shows a wound dressing (1) and a wound bandage (9) according to a first embodiment,

FIG. 2 shows a wound dressing (1) and a wound bandage (9) according to a second embodiment,

FIG. 3 shows schematic sketches of arc-shaped incisions (3), and

FIG. 4 shows schematic sketches of arc-shaped incisions (3), and

FIG. 5 shows schematic sketches of a multiplicity of arc-shaped incisions (3).

### DETAILED DESCRIPTION

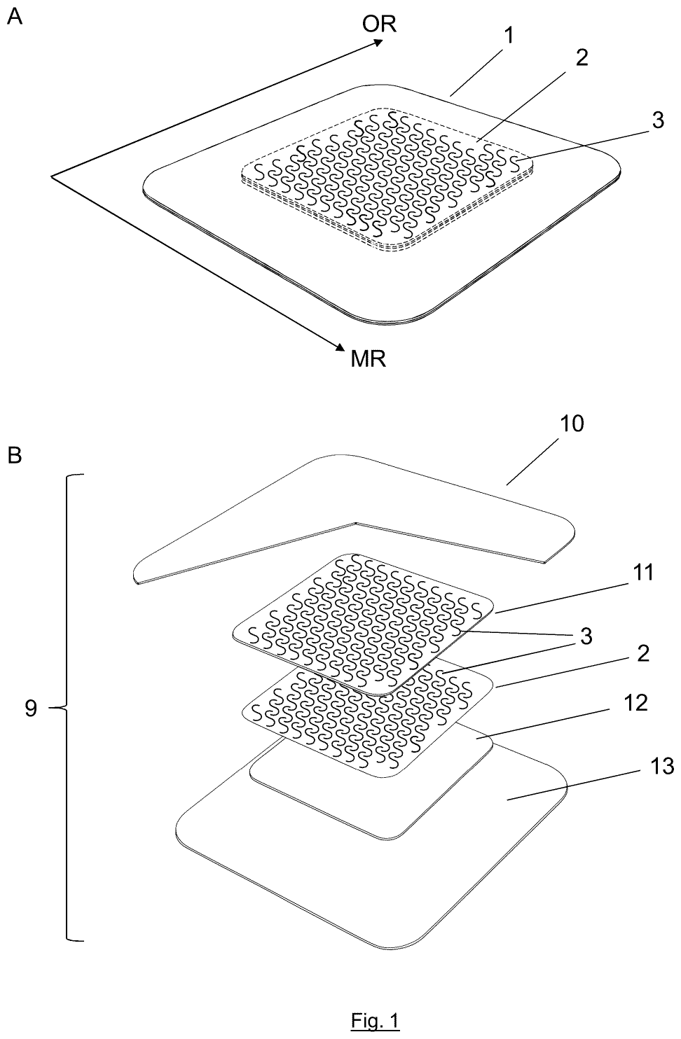

FIG. 1A shows a wound dressing (1) according to a first embodiment. The wound dressing (1) comprises a nonwoven (2) with a multiplicity of arc-shaped incisions (3). The incisions (3) are embodied in an S-shaped manner by way of example. The incisions (3) of the multiplicity of arc-shaped incisions (3) are distributed uniformly over the nonwoven (2). The planar extent of the wound dressing comprises a machine direction (MR) and a direction (OR) orthogonal thereto. The fibers of the nonwoven (2) have a desirable direction along the machine direction (MR), i.e. a majority of the fibers of the nonwoven (2) are aligned along the machine direction (MR). The fibers which reduce extensibility along the machine direction (MR) are severed by the arc-shaped incisions (3), as a result of which a wound dressing (1) according to embodiments with arc-shaped incisions (3) has excellent extensibility. The wound dressing (1) can include the nonwoven (2) or comprise further materials and/or layers. FIG. 1B shows a wound dressing (9) which comprises the nonwoven (2) and further comprises a carrier layer (10), a superabsorbent layer (11), a layer (12) comprising polyurethane and a wound contact layer (13). The superabsorbent layer (11) is arranged between the carrier layer (10) and the nonwoven (2). The wound dressing (1) includes the nonwoven (2) and is arranged between the superabsorbent layer (11) and the layer (12) comprising polyurethane. The layer (12) comprising polyurethane is arranged between the nonwoven (2) and the wound contact layer (13). The superabsorbent layer (11) comprises a multiplicity of arc-shaped incisions (3). In some embodiments, a pattern formed by the arc-shaped incisions (3) of the superabsorbent layer (11) is identical to a pattern formed by the arc-shaped incisions (3) of the nonwoven (2).

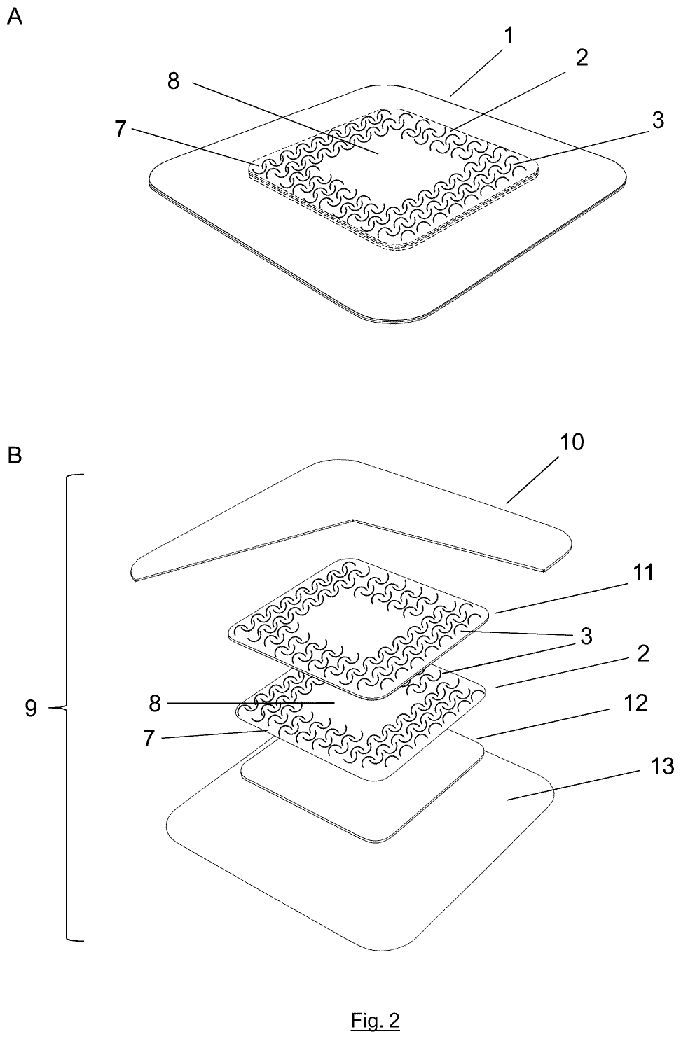

FIG. 2A shows a wound dressing (1) according to a second embodiment.

The wound dressing (1) comprises a nonwoven (2) with a multiplicity of arc-shaped incisions (3) in a peripheral region (7) of the nonwoven (2). The incisions (3) are embodied in a circle segment arc-shaped manner by way of example. A central region (8) of the nonwoven (2) has no arc-shaped incisions (3) here by way of example. The incisions (3) of the multiplicity of arc-shaped incisions (3) are distributed uniformly over the peripheral region (7) of the nonwoven (2). The wound dressing (1) can include the nonwoven (2) or comprise further materials and/or layers. FIG. 2B shows a wound dressing (9) which has a nonwoven (2) and further comprises a carrier layer (10), a superabsorbent layer (11), a layer (12) comprising polyurethane and a wound contact layer (13). The superabsorbent layer (11) is arranged between the carrier layer (10) and the nonwoven (2). The wound dressing (1) includes the nonwoven (2) and is arranged between the superabsorbent layer (11) and the layer (12) comprising polyurethane. The layer (12) comprising polyurethane is arranged between the nonwoven (2) and the wound contact layer (13). The superabsorbent layer (11) comprises a multiplicity of arc-shaped incisions (3). In some embodiments, a pattern formed by the arc-shaped incisions (3) of the superabsorbent layer (11) is identical to a pattern formed by the arc-shaped incisions (3) of the nonwoven (2).

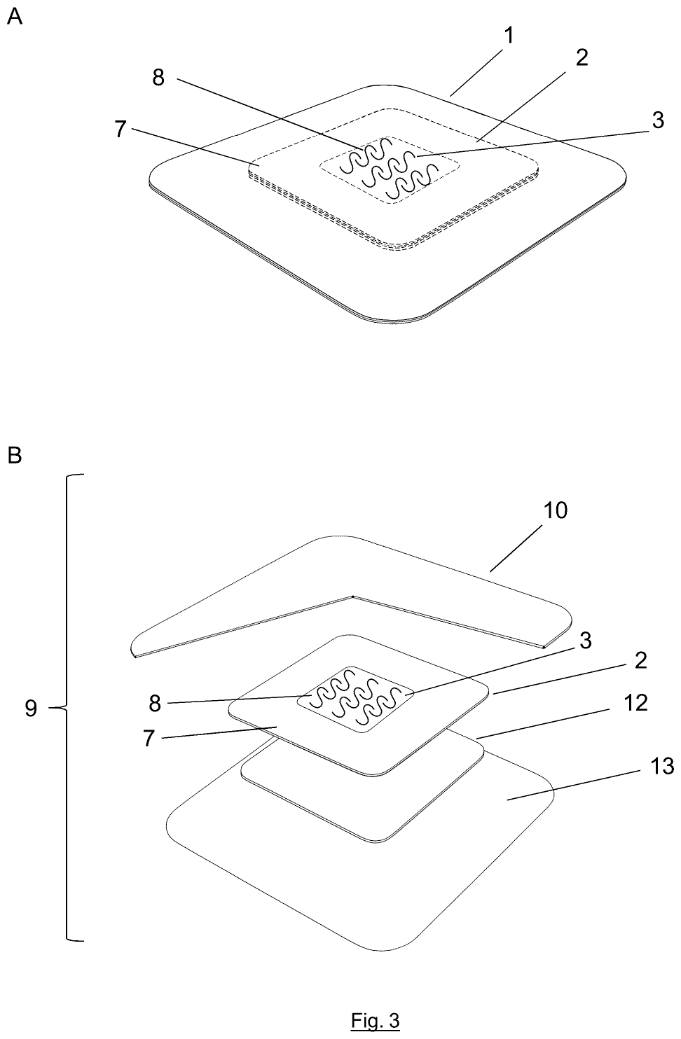

FIG. 3A shows a wound dressing (1) according to a third embodiment. The wound dressing (1) comprises a nonwoven (2) with a multiplicity of arc-shaped incisions (3) in a central region (8) of the nonwoven (2). The incisions (3) are embodied in an S-shaped manner by way of example. A peripheral region (7) of the nonwoven (2) has no arc-shaped incisions (3) here by way of example. The incisions (3) of the multiplicity of arc-shaped incisions (3) are distributed uniformly over the central region (8) of the nonwoven (2). FIG. 3B shows a wound dressing (9) which comprises a nonwoven (2) and further comprises a carrier layer (10), a layer (12) comprising polyurethane and a wound contact layer (13). The wound dressing (1) includes the nonwoven (2) and is arranged between the carrier layer (10) and the layer (12) comprising polyurethane. The layer (12) comprising polyurethane is arranged between the nonwoven (2) and the wound contact layer (13).

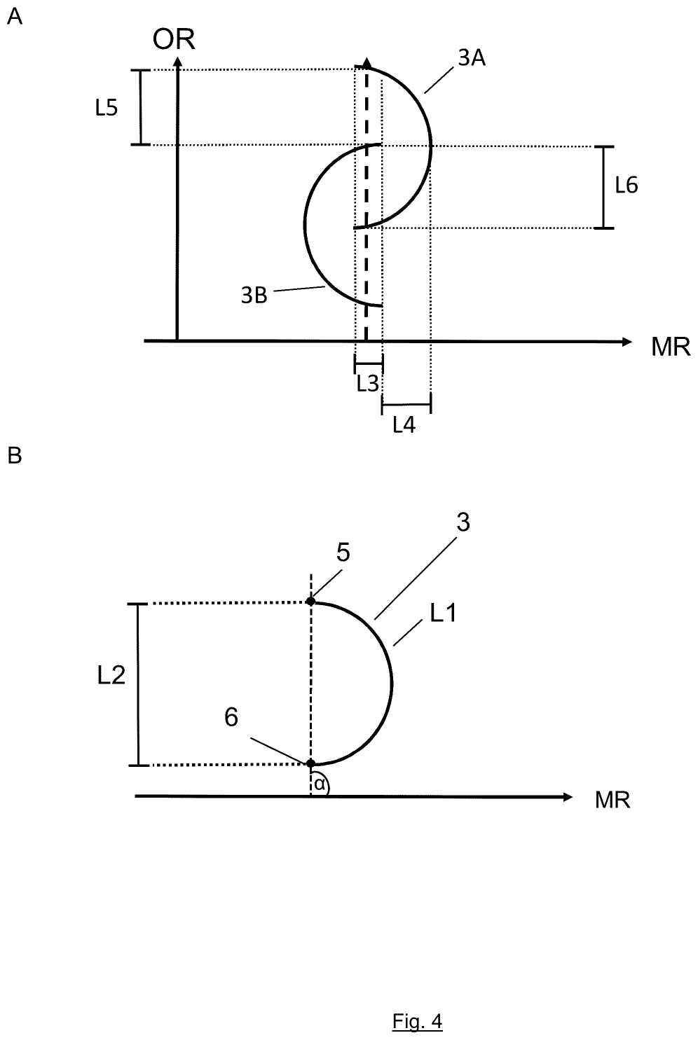

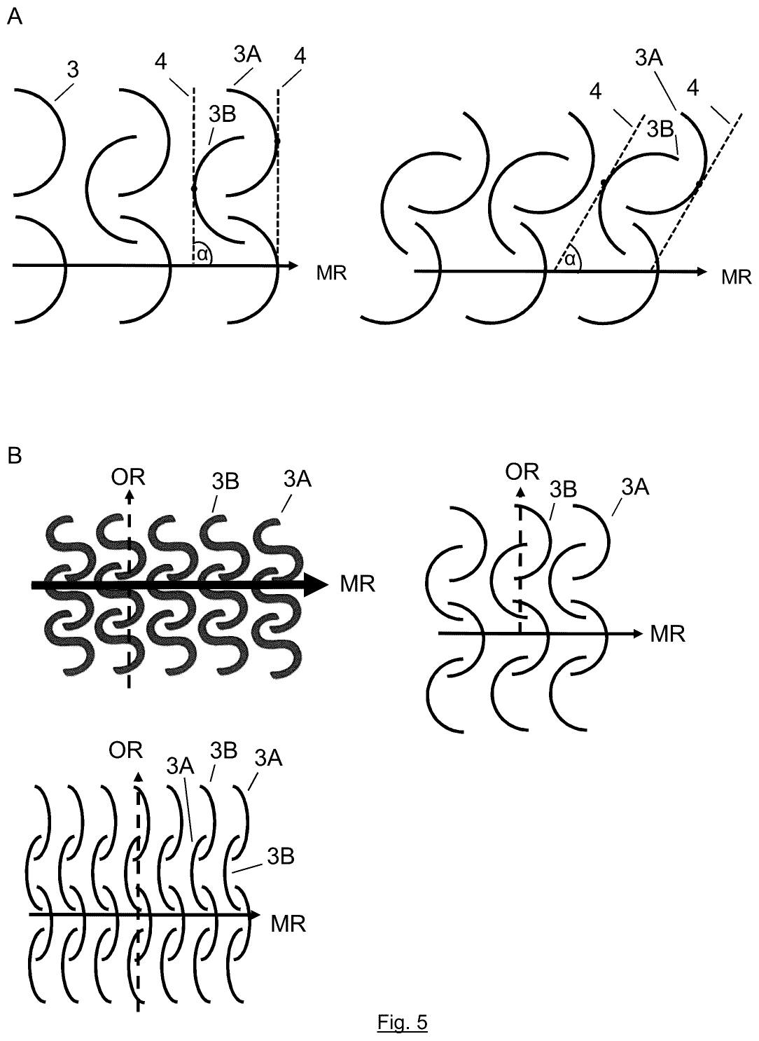

FIG. 4A shows two arc-shaped incisions (3A, 3B), spaced apart from one another, of a multiplicity of arc-shaped incisions (3), not illustrated, by way of example. The arc-shaped incisions (3A, 3B) are spaced apart along the machine direction (MR) and along the direction (OR) orthogonal to the machine direction (MR) along the planar extent of the wound dressing (1). A length (L4) of a spacing of two arc-shaped incisions (3A, 3B) spaced apart from one another along the machine direction (MR) is a minimum length between a point lying on the first incision (3A) and a point lying on the second incision (3B) along the machine direction (MR). The arc-shaped incisions (3) are designed such that the two arc-shaped incisions (3A, 3B) spaced apart from one another have an overlap in a length (L3), for example in a range of 0.1 mm to 5 mm, for example 0.2 mm to 2 mm, along the machine direction (MR). A length (L5) of a spacing of two arc-shaped incisions (3A, 3B) spaced apart from one another along the direction (OR) orthogonal to the machine direction is a minimum length between a point lying on the first incision (3A) and a point lying on the second incision (3B) along the orthogonal direction (OR). FIG. 4B shows an example arc-shaped incision (3) which has a length (L1) along the incision. A length (L2) between a first end point (5) of the arc-shaped incision (3) and a second end point (6) of the arc-shaped incision (3) may be embodied in a range of 0.5 mm to 12 mm, or more particularly 1 mm to 9 mm. A straight line running through the first end point (5) of the arc-shaped incision (3) and the second end point (6) of the arc-shaped incision (3) has an angle of 30° to 150°, or particularly 45° to 135°, or more particularly 60° to 120° to the machine direction (MR) of the nonwoven (2). FIG. 5A shows a multiplicity of arc-shaped incisions (3, 3A, 3B) spaced apart from one another. The tangents (4) applied to the arc-shaped incisions (3, 3A, 3B) each have an angle of 30° to 150°, or in some embodiments 45° to 135°, or in some embodiments 60° to 120° to the machine direction (MR) of the nonwoven (2). FIG. 5B shows three exemplary patterns of incisions (3A, 3B) which are formed by a multiplicity of incisions (3A, 3B) which are spaced apart from one another along the machine direction (MR) and along the direction (OR) orthogonal to the machine direction (MR). The nonwoven (2) and further layers of a wound dressing (9), for example a carrier layer (10), a superabsorbent layer (11), a layer (12) comprising polyurethane and/or a wound contact layer (13), can have corresponding patterns.

Embodiments may not be restricted to the example embodiments explained with reference to the drawing. Rather, embodiments are also contemplated in which arc-shaped incisions (3) with shapes other than those shown in FIG. 1, for example circle segment arc-shaped or wave-shaped incisions, are distributed uniformly over the entire area of the nonwoven (2), including the peripheral region (7) and the central region (8). Furthermore, embodiments are also contemplated in which arc-shaped incisions (3) with shapes other than those shown in FIG. 2, for example S-shaped or wave-shaped incisions, are distributed over the peripheral region (7) of the nonwoven (2). Furthermore, embodiments are also contemplated in which arc-shaped incisions (3) with shapes other than those shown in FIG. 3, for example circle segment arc-shaped or wave-shaped incisions, are distributed over the central region (8) of the nonwoven (2).

### REFERENCE SIGNS LIST

1 Wound dressing
2 Nonwoven
3 Arc-shaped incision
3A, 3B Arc-shaped incisions spaced apart from one another
4 tangent
5 First end point of the arc-shaped incision
6 Second end point of the arc-shaped incision
7 Peripheral region
8 Central region
9 Wound bandage
10 Carrier layer
11 Superabsorbent layer
12 Layer comprising polyurethane
13 Wound contact layer
MR Machine direction
OR Direction orthogonal to the machine direction
L1 Length of the arc-shaped incision
L2 Length between a first end point and a second end point of the arc-shaped incision
L3 Length of an overlap along the machine direction
L4 Length of a spacing along the machine direction
L5 Length of a spacing along the direction orthogonal to the machine direction

1-31. (canceled)

32. A wound dressing comprising:
a nonwoven fabric layer that includes a plurality of fibers;
wherein a majority of the fibers are aligned in a machine direction (MR) of the nonwoven fabric layer;
wherein the nonwoven fabric layer has at least one arc-shaped incision;
wherein a radius of curvature of the at least one arc-shaped incision is between 3 millimeters (mm) and 10 mm;
wherein a tangent applied to the arc-shaped incision has an angle of between 30 degrees (°) and 150°, preferably 45° to 135°, more preferably 60° to 120° to the MR of the nonwoven fabric layer;
wherein the arc-shaped incision is doubly rotationally symmetrical with respect to an axis that runs perpendicular to a planar extension of the wound dressing; and
wherein the arc-shaped incision is an S-shaped incision or a wave-shaped incision.

33. The wound dressing of claim 32, wherein the arc-shaped incision has a length in a range of between 1 mm to 15 mm.

34. The wound dressing of claim 32, wherein a length between a first end point of the arc-shaped incision and a second end point of the arc-shaped incision is between 0.5 mm and 12 mm.

35. The wound dressing of claim 32, wherein a straight line that runs through a first end point of the arc-shaped incision and a second end point of the arc-shaped incision has an angle of between 30° and 150° to the MR of the nonwoven fabric layer.

36. The wound dressing of claim 32, wherein the nonwoven has a multiplicity of arc-shaped incisions that are spaced apart from one another.

37. The wound dressing of claim 36, wherein at least two arc-shaped incisions, spaced apart from one another, of the multiplicity of arc-shaped incisions have a spacing of between 0.5 mm and 10 mm from one another.

38. The wound dressing of claim 36, wherein at least two arc-shaped incisions, spaced apart from one another, of the multiplicity of arc-shaped incisions have a spacing of between 0.5 mm and 10 mm between one another as measured along the MR of the nonwoven fabric layer.

39. The wound dressing of claim 36, wherein at least two arc-shaped incisions, spaced apart from one another, of the multiplicity of arc-shaped incisions have a spacing in a range of between 0.5 mm to 10 mm between one another as measured along a direction (OR) orthogonal to the MR.

40. The wound dressing of claim 36, wherein at least two arc-shaped incisions, spaced apart from one another, of the multiplicity of arc-shaped incisions have an overlap of between 0.1 mm and 5 mm as measured along the MR.

41. The wound dressing of claim 36, wherein respective arc-shaped incisions of the multiplicity of arc-shaped incisions have an identical shape to one another.

42. The wound dressing of claim 36, wherein the nonwoven fabric layer has a peripheral region and a central region, and wherein the peripheral region or the central region has the multiplicity of arc-shaped incisions.

43. The wound dressing of claim 36, wherein the arc-shaped incisions of the multiplicity of arc-shaped incisions are distributed uniformly over the nonwoven fabric layer.

44. The wound dressing of to claim 36, wherein the nonwoven fabric layer comprises a superabsorbent polymer.

45. The wound dressing of claim 44, wherein the superabsorbent polymer is embodied in particulate or fibrous form.

46. A wound bandage comprising
a wound dressing that includes:
a nonwoven fabric layer that includes a plurality of fibers;
wherein a majority of the fibers are aligned in a machine direction (MR) of the nonwoven fabric layer;
wherein the nonwoven fabric layer has at least one arc-shaped incision;
wherein a radius of curvature of the at least one arc-shaped incision is between 3 millimeters (mm) and 10 mm;
wherein a tangent applied to the arc-shaped incision has an angle of between 30 degrees (°) and 150°, preferably 45° to 135°, more preferably 60° to 120° to the MR of the nonwoven fabric layer;
wherein the arc-shaped incision is doubly rotationally symmetrical with respect to an axis that runs perpendicular to a planar extension of the wound dressing; and
wherein the arc-shaped incision is an S-shaped incision or a wave-shaped incision.

47. The wound bandage of claim 46, wherein the wound bandage further comprises a carrier layer, a superabsorbent layer, a layer comprising polyurethane, or a wound contact layer.

48. The wound bandage of claim 47, wherein the carrier layer is waterproof.

49. The wound bandage of claim 47, wherein the superabsorbent layer includes a superabsorbent polymer.

50. The wound bandage of claim 47, wherein the layer comprising polyurethane comprises polyurethane foam.

51. The wound bandage of claim 47, wherein the wound contact layer comprises silicone.

52. The wound bandage of claim 47, wherein the superabsorbent layer, the layer comprising polyurethane, or the wound contact layer comprise at least one arc-shaped incision.
